(19) United States
(12) Patent Application Publication
(10) Pub. No.: US 2026/0026759 A1
(43) Pub. Date: Jan. 29, 2026

(54) MOTION DETECTION SYSTEM AND MOTION DETECTION METHOD

(71) Applicant: TOYOTA JIDOSHA KABUSHIKI KAISHA, Toyota-shi (JP)

(72) Inventors: Makoto KOBAYASHI, Nisshin-shi (JP); Hidetaka SASAKI, Nagoya-shi (JP)

(73) Assignee: TOYOTA JIDOSHA KABUSHIKI KAISHA, Toyota-shi (JP)

(21) Appl. No.: 19/262,753

(22) Filed: Jul. 8, 2025

(30) Foreign Application Priority Data
Jul. 23, 2024 (JP) 2024-117367

(51) Int. Cl. A61B 5/00; A61B 5/11; G01P 21/00; G06F 3/01

(52) U.S. Cl. CPC A61B 5/744; A61B 5/1124; G01P 21/00; G06F 3/011; A61B 2560/0223; A61B 2562/0223

(57) ABSTRACT

A motion detection system includes: a sensor configured to be attached to a user and to detect a motion of the user; a display unit configured to display an avatar that moves in conjunction with an output of the sensor, and a determination unit configured to determine that the sensor is not properly prepared when the displayed avatar is deviated from a reference state although the user is in the reference state.

### CROSS-REFERENCE TO RELATED APPLICATION

This application claims priority to Japanese Patent Application No. 2024-117367 filed on Jul. 23, 2024. The disclosure of the above-identified application, including the specification, drawings, and claims, is incorporated by reference herein in its entirety.

### BACKGROUND

### 1. Technical Field

The present disclosure relates to motion detection systems and motion detection methods.

### 2. Description of Related Art

Japanese Unexamined Patent Application Publication No. 2022-34450 (JP 2022-34450 A) describes a motion state monitoring system that can suitably manage measurement results according to the attachment direction of a sensor.

### SUMMARY

However, J P 2022-34450 A does not disclose determining that the sensor is not properly prepared when a displayed avatar is deviated from a reference state. An object of the present disclosure is to provide a motion detection system and a motion detection method in which a determination unit is configured to determine that a sensor is not properly prepared when a displayed avatar is deviated from a reference state although a user is in the reference state.

A motion detection system of the present disclosure includes: a sensor configured to be attached to a user and to detect a motion of the user; a display unit configured to display an avatar that moves in conjunction with an output of the sensor; and
a determination unit configured to determine that the sensor is not properly prepared when the displayed avatar is deviated from a reference state although the user is in the reference state.

A motion detection method of the present disclosure includes:
attaching a sensor to a user and detecting a motion of the user;
displaying, on a display unit, an avatar that moves in conjunction with an output of the sensor; and
determining, by a determination unit, that the sensor is not properly prepared when the displayed avatar is deviated from a reference state although the user is in the reference state.

According to the present disclosure, it is possible to properly prepare for sensing.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a schematic configuration diagram of a training assist system according to a first embodiment;

FIG. 2 is a diagram for describing an example of attachment of a sensor of the measuring instrument according to the first embodiment;

FIG. 3 is a diagram for describing an initial reference direction according to the first embodiment;

FIG. 4 is a block diagram illustrating an example of a configuration of the training assist system according to the first embodiment;

FIG. 5 is a flowchart illustrating an example of a processing procedure of the motion state monitoring device according to the first embodiment;

FIG. 6 is a diagram illustrating an example of a display screen before the start of measurement of the display unit according to the first embodiment;

FIG. 7 is a diagram illustrating an example of a display screen at the end of measurement of the display unit according to the first embodiment;

FIG. 8 is a diagram illustrating an example of a data structure of an arithmetic processing table according to a second embodiment;

FIG. 9 is a flowchart illustrating an example of a processing procedure of the motion state monitoring device according to a third embodiment;

FIG. 10 is a schematic configuration diagram of a computer according to the embodiment; and

FIG. 11 is a display example of an avatar according to the first embodiment.

### DETAILED DESCRIPTION OF EMBODIMENTS

Hereinafter, the present disclosure will be described through embodiments, but the disclosure according to the claims is not limited to the following embodiments. Moreover, all of the configurations described in the embodiments are not necessarily indispensable as means for solving the issue. For clarity of explanation, the following description and the drawings are omitted and simplified as appropriate. In the drawings, the same elements are denoted by the same reference numerals.

### First Embodiment

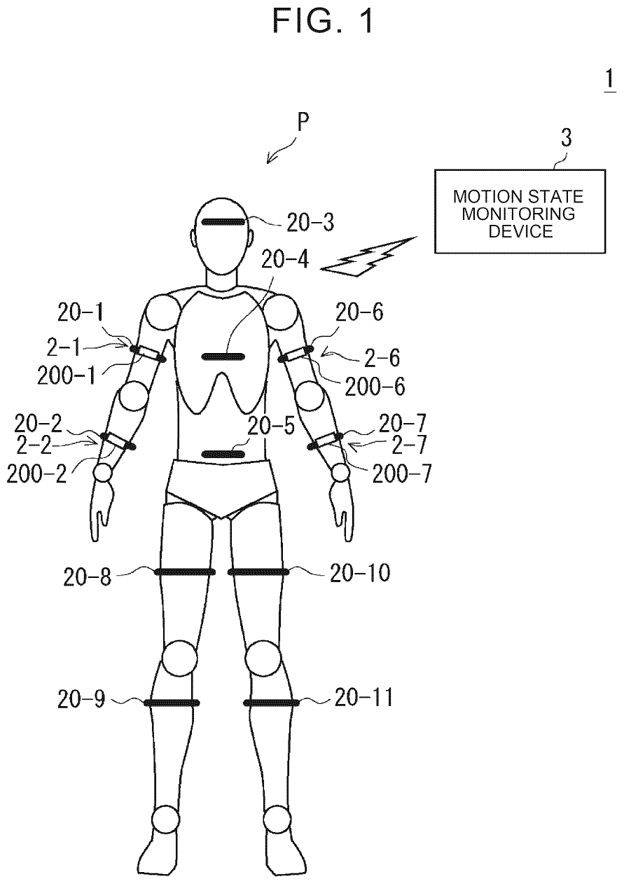

First, a first embodiment of the present disclosure will be described with reference to FIGS. 1 to 7 and 11.

FIG. 1 is a schematic configuration diagram of a training assist system 1 according to a first embodiment. The training assist system 1 is a computer system that assists in training by measuring a motor function of a subject P such as a rehabilitation trainee or an elderly person, and analyzing, evaluating, and managing a measurement result. The subject P performs the exercise test by attaching the sensor to a part of the body. For example, the exercise test is a motor function test in which a motion state of a target part is measured and a motor function is measured when the subject P performs a designated motion.

Hereinafter, the designated motion may be referred to as a monitoring target motion. The motion to be monitored is determined corresponding to a part of the body, and examples thereof include shoulder flexion extension, shoulder adduction/abduction, shoulder internal/external rotation, neck flexion extension, neck rotation, elbow flexion extension, hip joint internal/external rotation, forearm rotation internal/external/external, chest/lumbar flexion, and the like. When the target part is either the right or left, the monitoring target motion may be determined by distinguishing between the right and left. As a target part, one or more parts may be associated with one monitoring target motion, and the same part may be associated with different monitoring target motions.

As shown in this figure, the training assist system 1 includes a measuring instrument 2 and an motion state monitoring system (hereinafter referred to as motion state monitoring device) 3. The motion state monitoring system may be referred to as a motion detection system. The motion state monitoring device may be referred to as a motion detection device. Further, the motion state monitoring method may be referred to as a motion detection method.

The measuring instrument 2 is a measuring device that measures a moving direction and a moving amount. In the first embodiment, the measuring instrument 2 includes an acceleration sensor and an angular velocity sensor, and measures its own acceleration and angular velocity. Specifically, the measuring instrument 2 may include a three-axis acceleration sensor and a three-axis angular velocity sensor. Here, the measuring instrument 2 measures the displacement in the directions of three axes, namely the X, Y, and Z-axes, and the rotation angle about the three axes. The measurement axis is not limited to the three axes, and may be two axes or less. The measuring instrument 2 may include a geomagnetic sensor that detects geomagnetism and measures a direction in which the user is facing the geomagnetism.

The measuring instrument 2 is communicably connected to the motion state monitoring device 3. In the first embodiment, the communication between the measuring instrument 2 and the motion state monitoring device 3 is short-range radio communication such as Bluetooth (registered trademark), NFC (Near Field Communication), and ZigBee. However, the present disclosure is not limited thereto, and the communication may be wireless communication via a network such as a wireless LAN (Local Area Network). The communication may be wired communication via a network including the Internet, LAN, WAN (Wide Area Network), or a combination thereof.

The measuring instrument 2 includes a sensor 200 and an attachment mechanism of the sensor 200. The sensor 200 is attached to the attachment position 20 on the target part of the body of the subject P via the attachment mechanism. In order to cope with measurement of various monitoring target motions, each of the plurality of sensors 200 is associated with each of the body parts of the subject P and can be attached to the associated parts. In this figure, the attachable parts are shown at attachment positions 20-1, 20-2, . . . , and 20-11, each associated with sensors 200-1, 200-2, . . . , and 200-11. For example, the attachment positions 20-1, 20-2, . . . , and 20-11 are called right upper arm, right forearm, head, chest (trunk), lumbar region (pelvis), left upper arm, left forearm, right thigh, right lower leg, left thigh, and left lower leg, respectively. The association between the attachment position 20 and the sensor 200 is performed by pairing between the sensor 200 and the motion state monitoring device 3 in advance, and the identification (ID) of the attachment position 20 and ID of the sensor 200 are associated with each other on the application of the motion state monitoring device 3.

In the first embodiment, the attachment position 20 used in the exercise test is selected from the attachment positions 20-1 to 20-11 in accordance with the monitoring target motion selected by the user. The user is a user who uses the motion state monitoring device 3, and is, for example, the subject P himself or a staff who performs an exercise test. Then, the subject P or the staff attaches the sensor 200 (2-1, 2-2, 2-6, and 2-7 in this figure) associated with the selected attachment position 20 (20-1, 20-2, 20-6, and 20-7 in this figure) of the body of the subject P, and starts the exercise test.

Although a plurality of sensors 200 each associated with each of the plurality of attachment positions 20 is prepared, the number of attachment positions 20 to be prepared may be 1, and the number of sensors 200 to be prepared may be 1.

In response to the start of the exercise test, the sensor 200 starts measurement and transmits sensing information to the motion state monitoring device 3. The sensing information may include acceleration information, angular velocity information, or quaternion information. In addition, the sensing data may include components in the respective measurement axial directions (X, Y, and Z-axis directions). Then, the sensor 200 stops the measurement in response to the completion of the exercise test.

The motion state monitoring device 3 is a computer device that monitors a motion state of a target part of the body of the subject P during an exercise test, and analyzes, evaluates, and manages information related to the motion state. Specifically, the motion state monitoring device 3 may be a personal computer, a notebook computer, a mobile phone, a smartphone, a tablet, or other data communication terminal devices that can input and output data. The motion state monitoring device 3 may be a server computer. In the first embodiment, the motion state monitoring device 3 is described as a tablet terminal.

The motion state monitoring device 3 is used by the user during an exercise test and before and after an exercise test. The motion state monitoring device 3 receives the selection of the monitoring target motion from the user, and notifies the user of the attachment position 20 corresponding to the target part. In response to the start or end of the exercise test, the motion state monitoring device 3 transmits a request to start measurement or stop measurement to the sensor 200. In response to receiving the sensing information from the sensor 200, the motion state monitoring device 3 outputs the sensing-related information as a measurement result. Here, the sensing-related information indicates information related to the sensing information, may include the sensing information itself, and may be information obtained by performing various conversion processes on the sensing information. Further, the information regarding the motion state described above is information based on the sensing-related information, and may include the sensing-related information itself.

The motion state monitoring device 3 may be communicably connected to an external server (not shown) via a network. The external server may be a computer device or a cloud server on the Internet. In this case, the motion state monitoring device 3 may transmit the sensing-related information or the information regarding the motion state of the subject P held by itself to the external server.

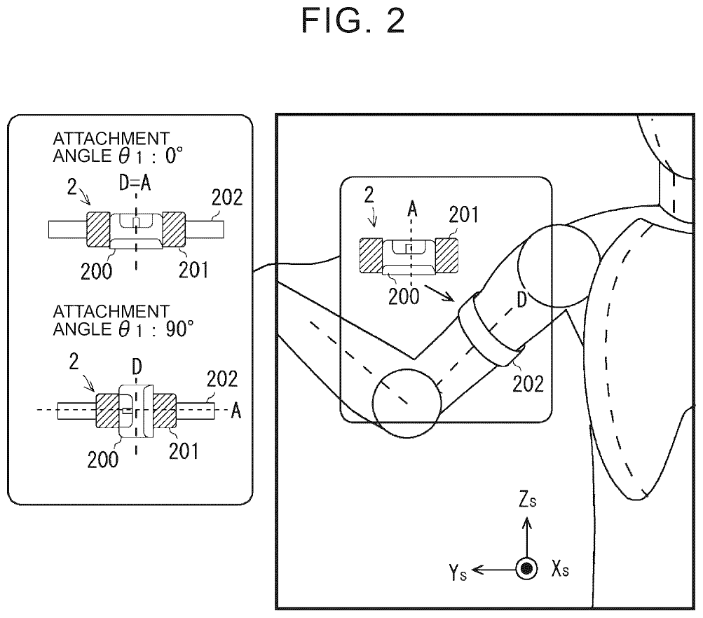

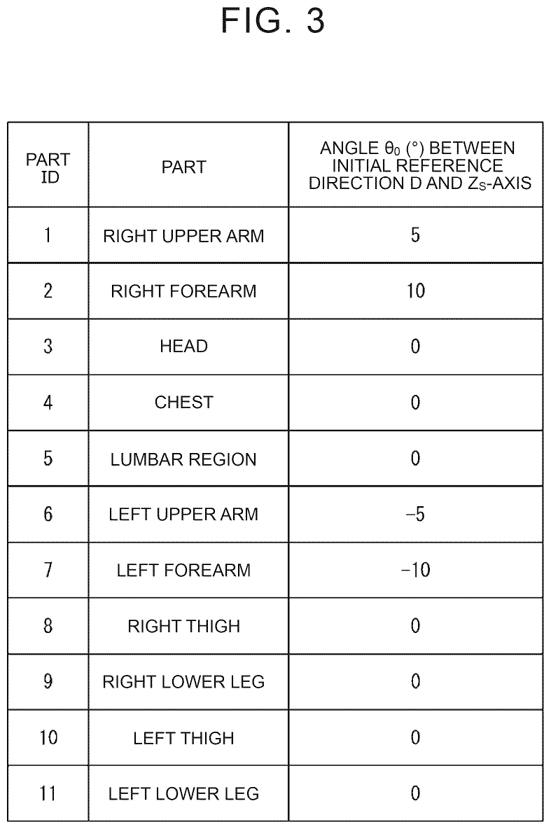

The attachment of the sensor 200 of the measuring instrument 2 according to the first embodiment will now be described with reference to FIGS. 2 and 3. FIG. 2 is a diagram for describing an example of attachment of the sensor 200 of the measuring instrument 2 according to the first embodiment.

As shown in FIG. 2, the measuring instrument 2 includes a sensor 200, an attachment pad 201 as an attachment mechanism (attachment tool), and a strip-shaped band 202. The sensor 200 is connected to a band 202 attached to the target part via an attachment pad 201. Thus, the sensor 200 is attached to the attachment position 20 of the target part. The connection mechanism (connector) between the sensor 200 and the band 202 is not limited to the attachment pad 201, and may be a fastener such as a hook or a snap, or a hook-and-loop fastener.

The attachment direction of the sensor 200 will be described. The attachment direction of the sensor 200 is the attachment direction of the sensor 200 with respect to the reference direction D. In the first embodiment, the reference direction D is a direction in which the attachment direction does not relatively change even if the target part is moved during the monitoring target motion. That is, the reference direction D is a direction that changes in conjunction with the absolute direction of the sensor 200 during the monitoring target motion. Here, the “absolute direction” is a direction with respect to the gravitational direction or the horizontal direction, and may be, for example, a direction defined by a coordinate system (X_{S}, Y_{S}, Z_{S}) with respect to the subject P. The X_{S}-axis is a horizontal axis in the front-rear direction with respect to the subject P, the Y_{S}-axis is a horizontal axis in the left-right direction with respect to the subject P, and the Z_{S}-axis is a vertical axis in the gravity direction.

In FIG. 2, the reference direction D is defined as the axial direction of the band 202 attached to the target part. The attachment direction indicates a relative direction of the sensor 200 with respect to the reference direction D, which is an axial direction, and is specifically determined based on an angle (referred to as attachment angle) θ₁ formed by the reference direction D and the measurement axis A of the sensor. The measurement axis A may be predetermined and may be, for example, any of the X, Y and Z axes of the sensor coordinate system. For example, as shown in FIG. 2, when the attachment angle θ₁ is 0°, the sensor 200 is attached so that the measurement axis A is parallel to the reference direction D. When the attachment angle θ₁ is 90°, the sensor 200 is attached such that the measurement axis A is perpendicular to the reference direction D. Note that the attachment angle θ₁ is not limited to 0° and 90°.

In the first embodiment, the reference direction D can be defined according to the target part. For example, in the case where the band 202 is attached to the target part, there is a certain preferable attachment direction for each target part. For example, when the target part is an arm, the band 202 is preferably attached such that the reference direction D is substantially parallel to the axial direction of the arm (that is, the extending direction of the arm) from the viewpoint of case of attachment and case of movement. On the other hand, it is difficult to attach the band 202 to the arm so that the reference direction D is substantially perpendicular to the axial direction of the arm. Therefore, the axial direction of the band 202 as the reference direction D can be defined in advance according to the target part.

In FIG. 2, the sensor 200 is attached to the target part using the band 202, but the band 202 may be omitted. At this time, the sensor 200 may be attached to a garment or the skin via the attachment pad 201. Also in this case, the reference direction D is a direction defined in advance according to the target part, such as the axial direction of the target part.

In the first embodiment, the attachment mechanism of the measuring instrument 2 includes a change mechanism that changes the attachment direction of the sensor 200. The changing mechanism may be any mechanism that enables the attachment direction of the sensor 200 to be changed. For example, when the sensor 200 has an adhesive surface on which the attachment pad 201 can be used repeatedly, the attachment direction is freely changed. When the sensor 200 is attached to a target part using a connector between a belt and clothes, the attachment direction may be changed using a knob or the like interlocked with the connector after the sensor 200 is attached so as to substantially coincide with the reference direction D. In addition, when the sensor 200 is attached using a connector having a shape capable of holding the sensor 200 in a plurality of attachment directions, the sensor 200 may be attached in one attachment direction selected from the plurality of attachment directions.

In the first embodiment, the reference direction D can be specifically determined in advance according to the target part in the initial state, that is, in the stationary state. FIG. 3 is a diagram for explaining an initial reference direction D according to the first embodiment. As shown in this figure, the absolute direction of the initial reference direction D is defined corresponding to each part. In this diagram, the absolute direction of the initial reference direction D is expressed using the angle θ₀ formed between the Z_{S}-axis. The angle θ₀ may be determined based on the human mean skeleton. In the present embodiment, the initial reference direction D of the upper arm faces outward with respect to the Z_{S}-axis, and for example, the angle θ₀ of the right upper arm may be defined as 5°. Also, the initial reference direction D of the forearm is further outward with respect to the Z_{S}-axis than the upper arm, and for example, the angle θ₀ of the right forearm may be determined to be 10°. Note that the angle θ₀ for each part may be determined for each subject P on the basis of attribution data such as age, gender, height, or weight of the subject P. Even in the case where the initial reference direction D changes according to the target part as described above, since the initial reference direction D is specifically determined, at least the initial attachment direction can be converted into an absolute direction which is a unique index for the subject P.

As described above, the sensor 200 according to the first embodiment is configured so that the attachment direction can be changed. Therefore, the user can freely set the attachment direction of the sensor 200, thereby improving convenience. In addition, by setting the sensor 200 in a suitable direction, the accuracy of the measurement result is improved.

Hereinafter, the attachment direction with respect to the reference direction D is simply referred to as the “attachment direction”.

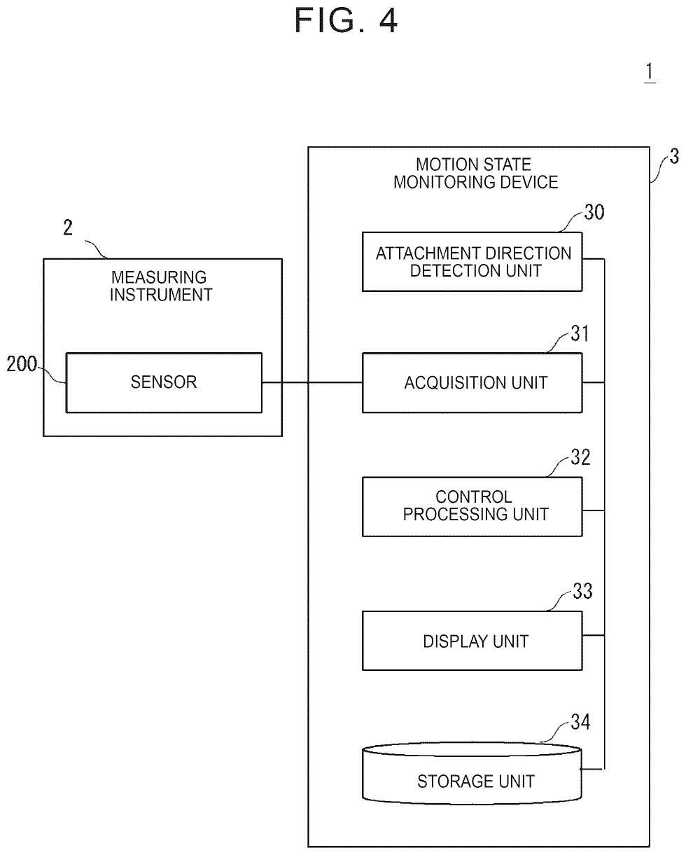

FIG. 4 is a block diagram illustrating an example of a configuration of the training assist system 1 according to the first embodiment. As described above, the training assist system 1 includes the measuring instrument 2 and the motion state monitoring device 3, and the measuring instrument 2 includes the sensor 200. In this figure, the sensor 200 is the sensor 200 associated with the attachment position 20 selected based on the monitoring target motion out of the prepared sensors 200-1 to 200-11. It is assumed that the sensor 200 is paired with the motion state monitoring device 3 in advance and is calibrated. The number of sensors 200 is not limited to one, and may be two or more.

The motion state monitoring device 3 includes an attachment direction detection unit 30, an acquisition unit 31, a control processing unit 32, a display unit 33, and a storage unit 34.

The attachment direction detection unit 30 detects the attachment direction of the sensor 200. For example, the attachment direction detection unit 30 may detect the attachment direction of the sensor 200 based on the output of the sensor 200 at the time of attachment. In this case, the attachment direction detection unit 30 calculates the attachment angle with respect to the Z_{S}-axis based on the information of the Z_{S}-axis acquired from the sensor 200 at the time of calibration and the angle information of the sensor 200 from the stationary state in the calibration to the time of attachment. In this way, the attachment direction detection unit 30 can detect the attachment direction of the sensor 200.

Further, for example, the attachment direction detection unit 30 may include an attachment direction detection sensor and an attachment direction detection mechanism separately disposed in the vicinity of each sensor 200. The attachment direction detection mechanism is configured such that a current flows in accordance with an angle between the measurement axis A of the sensor 200 and the reference direction D, and the attachment direction detection sensor detects the current. Then, the attachment direction is detected in accordance with the magnitude of the detected current. When the band 202 is used for attaching the sensor 200, the attachment direction detection sensor and the attachment direction detection mechanism may be disposed on the band 202. The attachment direction detection sensor and the attachment direction detection mechanism may be included in the measuring instrument 2, and the attachment direction detection unit 30 may acquire information on the attachment direction based on an output from the attachment direction detection sensor.

Further, for example, the attachment direction detection unit 30 may detect the attachment direction of the sensor 200 based on the captured image of the attached sensor 200. For example, the attachment direction detection unit 30 may include an attachment direction detection camera disposed in front of, behind, above, or the like of the subject P. Then, the attachment direction detection unit 30 may detect the attachment direction of the sensor 200 by capturing an image of the sensor 200 and performing image processing such as pattern matching on the captured image. The attachment direction detection camera may be included in the measuring instrument 2, and the attachment direction detection unit 30 may acquire an image from the attachment direction detection camera and acquire information on the attachment direction based on the image.

In addition, when the attachment direction of the sensor 200 can be adjusted by a knob or the like interlocked with the connector, the attachment direction detection unit 30 may detect the attachment direction based on the amount of movement of the knob. In the first embodiment, the attachment direction detection unit 30 detects an attachment direction of the sensor 200 in an initial state, that is, in a stationary state immediately before measurement. Then, the attachment direction detection unit 30 supplies information on the detected attachment direction to the control processing unit 32.

The acquisition unit 31 acquires sensing information of the sensor 200. In the first embodiment, the acquisition unit 31 receives and acquires sensing information from the sensor 200. However, the present disclosure is not limited thereto, and the acquisition unit 31 may indirectly acquire the sensing information from an external computer (not shown) that holds the sensing information. The acquisition unit 31 supplies the acquired sensing information to the control processing unit 32.

The control processing unit 32 controls each component of the sensor 200 and the motion state monitoring device 3. In addition, the control processing unit 32 executes tagging processing for associating the attachment direction of the sensor 200 with the sensing-related information in the attachment direction. Then, the control processing unit 32 outputs the sensing-related information after the tagging processing associated with the attachment direction of the sensor 200 via the output unit. In addition, the control processing unit 32 may store the sensing-related information after the tagging processing in the storage unit 34.

The display unit 33 is an example of an output unit, and is a display that displays sensing-related information supplied from the control processing unit 32. The display unit 33 displays an avatar based on a sensor attached to the user. The avatar is a character or a 3D that serves as a user's status in the virtual space. The avatar performs the same motion as the user, and can confirm the user's motion from various angles by playing back, slow displaying, or the like. In the first embodiment, the display unit 33 may be a touch panel configured together with an input unit (not shown). The output unit may include, instead of or in addition to the display unit 33, a sound output unit that outputs sensing-related information by voice, a data output unit that outputs the sensing-related information in a predetermined data format, or a transmission unit that transmits the sensing-related information to an external server or the like.

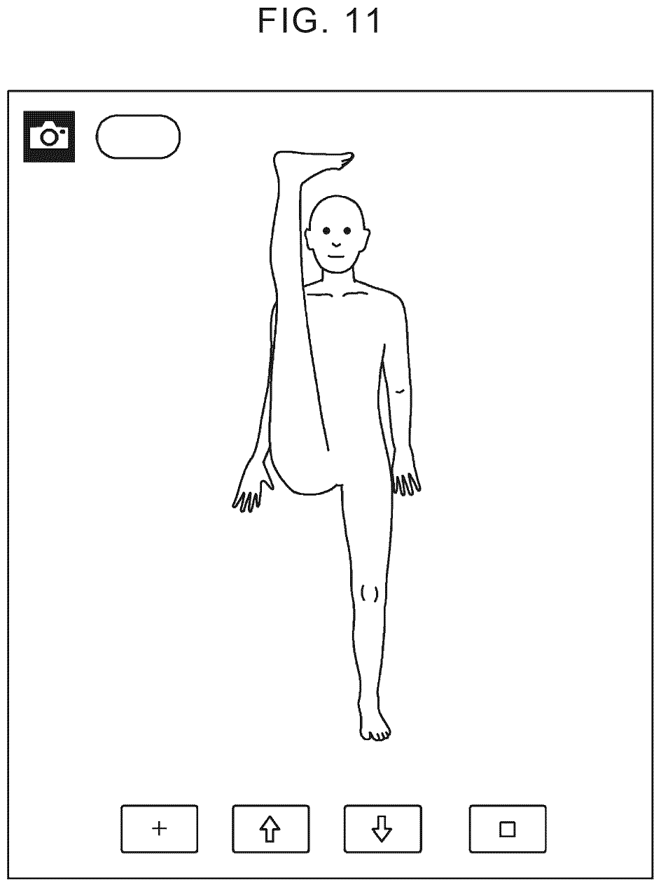

The control processing unit 32 includes a determination unit that determines that the sensor is not properly prepared when the displayed avatar is deviated from the reference state although the user is in the reference state. FIG. 11 shows an example of the display of an avatar immediately after the sensor 200 is attached to the user. As shown in FIG. 11, the displayed avatar being deviated from the reference state is a state in which the avatar is raising the right foot high when the user is in a relaxed standing position. When the relative positional relationship between the body parts of the displayed avatar that moves in conjunction with the output of the sensor attached to the user is deviated from the reference state such that the limb being twisted with respect to the torso, for example, it is determined that the sensor is not properly prepared. The sensor being not properly prepared indicates that the attachment position of the sensor is displaced or that the sensor needs to be recalibrated. Therefore, the deviation from the reference state occurs when the sensor is displaced by 180° etc. When the determination unit determines that the sensor is not properly prepared, the control processing unit 32 notifies by voice, text display on the display unit, highlighted display on the display unit, etc.

The storage unit 34 is a storage medium that stores information necessary for various processes of the motion state monitoring device 3. The storage unit 34 may store the sensing-related information after the tagging process, but this is not essential when the output unit includes a transmitter.

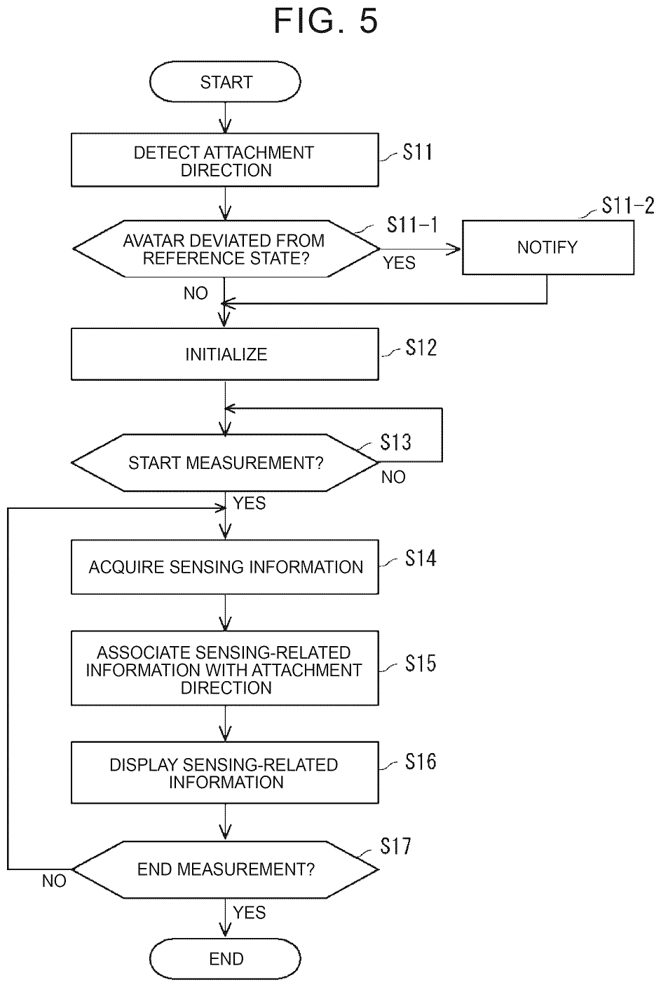

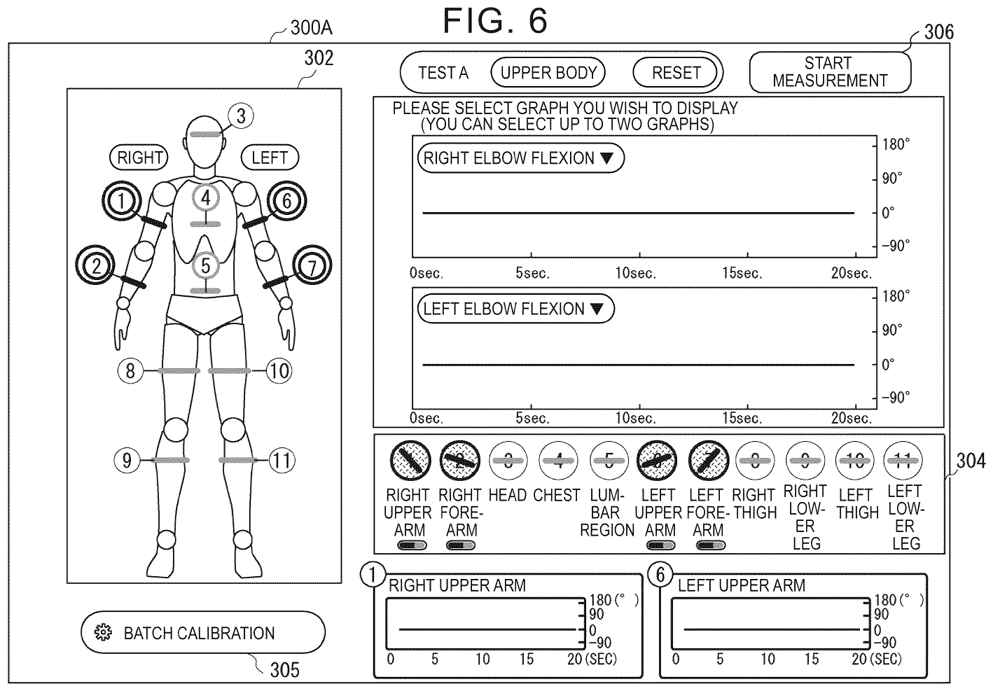

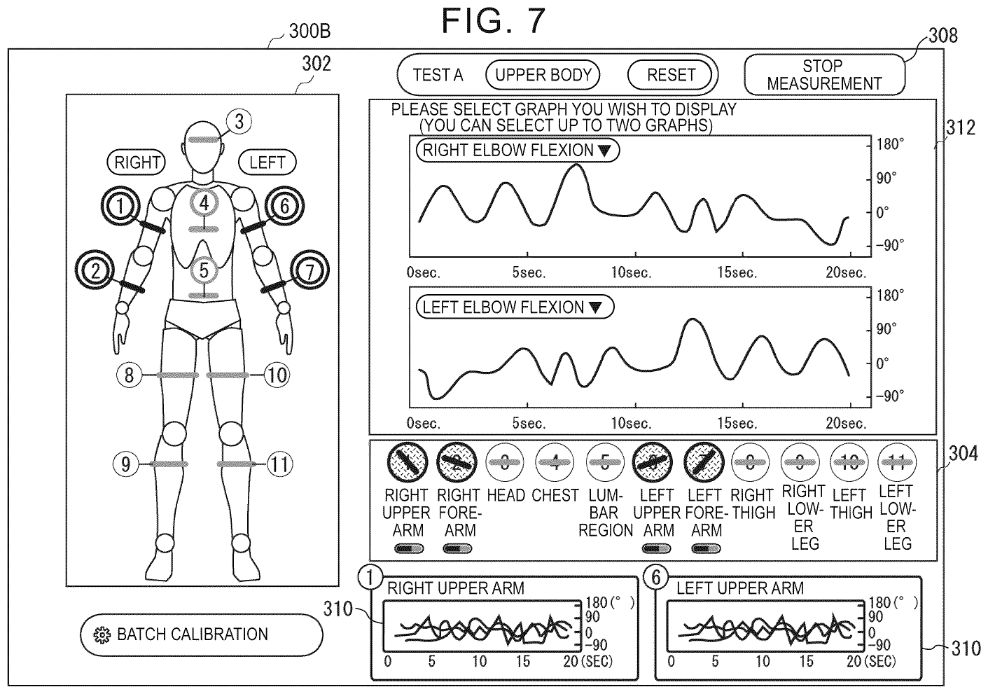

Next, an motion state monitoring method according to the first embodiment will be described with reference to FIGS. 6 and 7 as appropriate with reference to FIG. 5. FIG. 5 is a flowchart illustrating an example of a processing procedure of the motion state monitoring device 3 according to the first embodiment. FIG. 6 is a diagram illustrating an example of a display screen of the display unit 33 before the start of measurement according to the first embodiment. FIG. 7 is a diagram illustrating an example of a display screen at the end of measurement of the display unit 33 according to the first embodiment.

The step shown in FIG. 5 starts with the monitoring target motion being selected by the user, the attachment position 20 being determined based on the monitoring target motion, and the sensor 200 being attached to the attachment position 20 corresponding to the monitoring target motion. In the following example, the control processing unit 32 treats the sensing information as sensing-related information.

First, the attachment direction detection unit 30 of the motion state monitoring device 3 detects the attachment direction of the sensor 200 in response to the fact that the subject P and the sensor 200 are in a stationary state (S11). After the sensor 200 is calibrated, the control processing unit 32 determines whether the avatar displayed in the stationary state is deviated from the reference state (S11-1). When the avatar is deviated from the reference state (YES in S11-1), the control processing unit 32 notifies that the sensor is not properly prepared by voice, text-display on the display unit, or highlighted display on the display unit (S11-2). When notified, the user can reattach the sensor 200. The user may recalibrate the sensor 200. The control processing unit 32 then initializes the output value of the sensor 200 (S12). Specifically, the control processing unit 32 corrects the output value of the sensor 200 in the stationary state immediately before the measurement to 0. Even when calibration is performed, the sensor 200 cannot set an output error such as a drift error to 0, and the error increases according to the elapsed time. This step can therefore minimize the output error from the start to the end of measurement. However, if the output error is minor, this step may be omitted. The control processing unit 32 then determines whether to start the measurement by the sensor 200 (S13). When the measurement by the sensor 200 is to be started (Yes in S13), the process proceeds to S14. Otherwise (No in S13), the control processing unit 32 repeats the process shown in S13.

FIG. 6 shows a display image 300A before measurement is started as displayed by the display unit 33. The display image 300A includes a plurality of display areas 302 to 306.

Icon images representing a plurality of attachment positions 20 as attachment candidates for the sensor 200 are displayed in the display area 302. The icon image may be an avatar of the user. The attachment positions 20 corresponding to the selected measurement motion (positions shown by “1”, “2”, “6”, and “7” in the figure) may be highlighted in the display area 302. This allows the user to easily visually recognize the attachment positions 20, so that the exercise test can be smoothly performed.

When the user clicks an icon image representing an attachment position 20 in the display area 302, an image (not shown) indicating the attachment direction of the sensor 200 associated with this attachment position 20 is displayed. This allows the user to easily grasp the attachment direction of each sensor 200 through the image.

Rotation angles of the sensors 200-1, 200-2, . . . , and 200-11 associated with the attachment positions 20-1, 20-2, . . . , and 20-11 are displayed two-dimensionally in the display area 304. The rotation angle thus displayed dynamically changes in accordance with the movement of the sensor 200 that occurs in conjunction with the motion of the subject P. This allows the user to identify the sensor 200 that is powered off or the sensor 200 that is not operating properly via the display area 304 before starting the measurement. Alternatively, the attachment directions of the sensors 200-1, 200-2, . . . , and 200-11 associated with the attachment positions 20-1, 20-2, . . . , and 20-11 may be visually displayed in the display area 304. Therefore, the user can intuitively grasp the attachment direction of each sensor 200 via the display area 304.

When a plurality of sensors 200 is used for the exercise test, an input operation button for calibrating the plurality of sensors 200 at a time is displayed in the display area 305. This allows the user to easily request calibration of each of the plurality of sensors 200 via the display area 305.

An input operation button for starting the exercise test, that is, for starting the measurement by the sensor 200, is displayed in the display area 306. This allows the user to easily request to start the measurement by the sensor 200 via the display area 306.

In S14 shown in FIG. 5, the control processing unit 32 acquires sensing information from the sensor 200 via the acquisition unit 31. Then, the control processing unit 32 uses the sensing information as the sensing-related information, and assigns information on the attachment direction of the sensor 200 to the sensing-related information as tags, thereby associating the attachment direction with the sensing-related information (S15). The control processing unit 32 supplies the sensing-related information after the tagging processing to the display unit 33 and causes the information to be displayed (S16). Then, the control processing unit 32 determines whether to end the measurement by the sensor 200 (S17). When the measurement is to be ended (Yes in S17), the control processing unit 32 ends the process. Otherwise (No in S17), the process returns to S14.

In the above example, the motion state monitoring device 3 waits for the process of S12 and determines in S13 whether to start the measurement by the sensor 200. Alternatively, the motion state monitoring device 3 may perform the process of S12 in response to the determination that the measurement by the sensor 200 is to be started after the process of S11 (Yes in S13). In this case, the control processing unit 32 may proceed to S14 after, or in parallel with, executing the process of S12. When the measurement by the sensor 200 is not to be started (No in S13), the motion state monitoring device 3 may repeat the process shown in S13.

In addition, in the above-described example, the sensing information is used as the sensing-related information in the motion state monitoring device 3, but the sensing information subjected to various conversion processes may be used instead of or in addition to the sensing information. The converting process may include converting the quaternion data into rotation angles about the X, Y, and Z-axis. The rotation angle around the X_{S}-axis indicates the roll angle, the rotation angle around the Y_{S}-axis indicates the pitch angle, and the rotation angle around the Z_{S}-axis indicates the yaw angle. The control processing unit 32 calculates rotation angles about the X, Y, and Z-axes of the sensor coordinate system using the quaternion data, and converts the calculated rotation angles to a yaw angle, a roll angle, and a pitch angle. The conversion process may include a normalization process, a normalization process, or a synthesis process of the graph. In this case, the control processing unit 32 may assign information on the attachment direction of the sensor 200 as a tag to the sensing information after the conversion processing, instead of or in addition to S15, and associate the attachment direction with the sensing information after the conversion processing.

FIG. 7 shows a display image 300B at the time of completion of the measurement displayed by the display unit 33. The display image 300B includes a plurality of display areas 302 to 312. The display areas 302, 304 of the display image 300B are the same as the display areas 302, 304 of the display image 300A shown in FIG. 6.

The attachment direction of each sensor 200 used may be displayed in the vicinity of the icon image representing the attachment position 20 of the display area 302, or may be displayed in response to the user clicking on the icon image. As a result, the user can intuitively grasp the attachment direction of the sensor 200 used.

In the display area 308, an input operation button for terminating the exercise test, that is, stopping the measurement by the sensor 200 is displayed. Thus, the user can easily request to stop the measurement by the sensor 200 via the display area 308.

In the display area 310, sensing-related information of each sensor 200 used is displayed. In this figure, the rotation angles about X_{S}, Y_{S}, and Z_{S} axes based on the outputs of part of the sensors 200-1, 200-2, 200-6, and 200-7 used, namely the sensors 200-1, 200-6, are displayed in time series. Therefore, the display area 310 outputs sensing-related information associated with the attachment direction of the sensor 200 that has been used together with the display area 304 by display. As a result, the display area 310 allows the user to grasp the attachment condition and the measurement result in association with each other. Accordingly, the user can separately analyze, evaluate, or use the measurement result for each attachment condition.

In the display area 312, a motion state index of the target part for each of the monitored target motions is displayed. The motion state index is an index indicating a motion state of the target part in a case where the monitoring target motion is performed. The control processing unit 32 calculates a motion state index of the target part based on the sensing-related information of the sensor 200. For example, when the monitoring target motion is “right elbow flexion and extension”, the sensing-related information of the sensors 200-1, 200-2 at the attachment positions 20-1, 20-2 is used. In this case, the control processing unit 32 may calculate the motion state index based on the difference between the sensing-related information of the sensors 200-1, 200-2. Specifically, the control processing unit 32 calculates the three-dimensional rotation angle as the motion state index based on the difference between the quaternion information of the sensors 200-1, 200-2. In this case, the rotation angle is calculated in the order of Z-axis→Y-axis→X-axis, and is converted into the rotation angle around the X_{S}, Y_{S}, and Z_{S}-axes. The calculation order of the rotation angle may be determined in advance according to the monitoring target motion. In this figure, the display area 312 displays a time-series motion state index for a part of the monitoring target motions among the performed monitoring target motions.

As described above, according to the first embodiment, the motion state monitoring device 3 outputs the attachment direction of the sensor 200 and the measurement result in association with each other. Therefore, the motion state monitoring device 3 can suitably manage the measurement result according to the attachment direction of the sensor 200, thereby improving the convenience.

Further, since the motion state monitoring device 3 automatically detects the initial attachment direction of the sensor 200, the attachment direction at the time of attachment can be suitably set in accordance with the preference of the subject P or the staff, and the association with the measurement result can be facilitated.

### Second Embodiment

Next, a second embodiment of the present disclosure will be described. The second embodiment is characterized in that calculation processing according to the attachment direction is performed on the measurement result. The training assist system 1 according to the second embodiment has the same configuration and functions as those of the training assist system 1 according to the first embodiment, and thus description thereof will be omitted.

The control processing unit 32 of the motion state monitoring device 3 of the training assist system 1 executes arithmetic processing corresponding to the attachment direction with respect to the sensing information or the sensing-related information. The arithmetic processing here may be, for example, arithmetic processing that cancels or suppresses the influence of the attachment direction when the sensing-related information changes depending on the attachment direction even when the target part is moved in the same manner in the same monitoring target motion. In particular, when the control processing unit 32 calculates the rotation angles around the X-axis, the Y-axis, and the Z-axis using the quaternion information and converts the rotation angles to the rotation angles about the X_{S}-axis, the Y_{S}-axis, and the rotation angles about the Z_{S}-axis, it is necessary to convert four-dimensional vector data to three-dimensional data. In this calculation process, there is a problem that the obtained rotation angles differ depending on the order in which the rotation angles around the respective axes are calculated, and the results cannot be compared correctly. In order to suppress such an influence, it is preferable to determine the calculation order of the rotation angle in advance. Here, since the preferred calculation order of the rotation angle depends on the attachment direction of the sensor 200, it is effective to determine the calculation order corresponding to the attachment direction of the sensor 200.

Therefore, in the second embodiment, the control processing unit 32 executes the arithmetic processing using the arithmetic processing table 320 that defines the arithmetic processing mode according to the attachment direction. Then, the control processing unit 32 causes the output unit to output the calculation processing result in association with the initial attachment direction of the sensor 200.

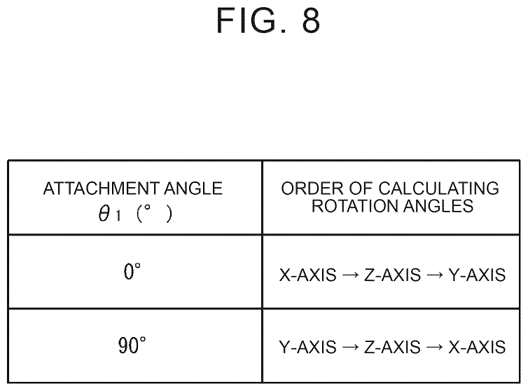

FIG. 8 is a diagram illustrating an example of a data structure of the arithmetic processing table 320 according to the second embodiment. As illustrated in the drawing, the arithmetic processing table 320 is a table that associates the attachment angle θ₁ with the order of calculating the rotation angles. For example, the arithmetic processing table 320 defines that, when the attachment angle θ₁ is 0°, the rotation angles about the axes are calculated in the order of X-axis→Z-axis→Y-axis. The arithmetic processing table 320 also defines that, when the attachment angle θ₁ is 90°, the rotation angles around the axes are calculated in the order of Y-axis→Z-axis→X-axis. By referring to the arithmetic processing table 320, the control processing unit 32 can easily execute preferable arithmetic processing according to the attachment direction.

The arithmetic processing table 320 determines the calculation order of the rotation angle according to the attachment direction of the sensor 200, but instead, the calculation order of the rotation angle may be determined according to the attachment direction and the target part or the monitoring target motion.

The arithmetic processing table 320 may include calculation parameters used in the calculation processing, instead of or in addition to the calculation order of the rotation angles. The calculation parameter may be a constant determined according to the attachment angle θ₁, and may include a predetermined function with the attachment direction θ₁ as a variable.

As described above, according to the second embodiment, the control processing unit 32 can easily compare and use a plurality of measurement results regardless of the attachment direction of the sensor 200. In the second embodiment as well, the same effects as those of the first embodiment are obtained.

### Third Embodiment

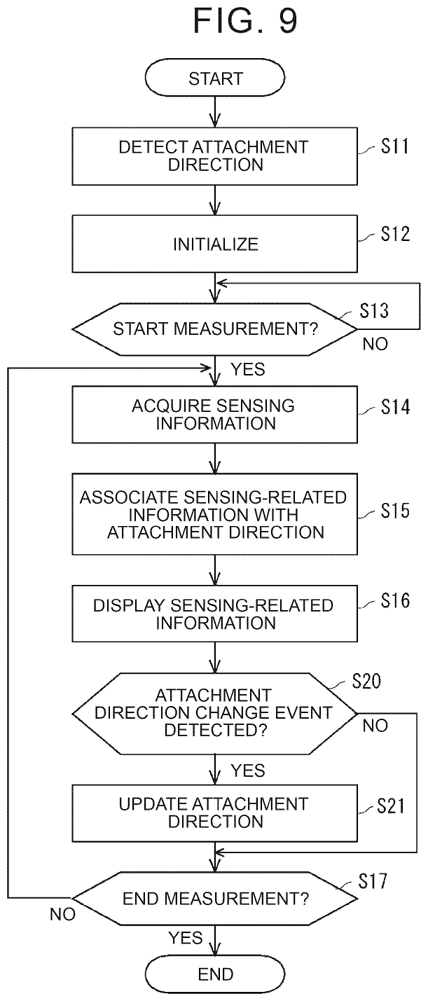

Next, a third embodiment of the present disclosure will be described with reference to FIG. 9. The third embodiment is characterized in that the attachment direction of the sensor 200 is detected not only in the initial state but also in the motion to be monitored. Since the motion state monitoring device 3 according to the third embodiment has the same configuration as that of the motion state monitoring device 3 according to the first or second embodiment, description thereof will be omitted. However, in the motion state monitoring device 3 according to the third embodiment, the attachment direction detection unit 30 detects the attachment direction during measurement of the sensor 200 in addition to the initial state. In the motion state monitoring device 3 according to the third embodiment, the control processing unit 32 outputs the sensing-related information after the event in association with the attachment direction after the event in response to the detection of the event in which the attachment direction changes during the measurement by the sensor 200.

FIG. 9 is a flowchart illustrating an example of a processing procedure of the motion state monitoring device 3 according to the third embodiment. The steps shown in this figure include S20 and S21 in addition to the steps shown in FIG. 5. Steps similar to those shown in FIG. 5 are denoted by the same symbols, and description thereof will be omitted. Although S11-1 and S11-2 are not shown, these steps may be included in FIG. 9.

In response to the display of the sensing-related information by the display unit 33 in S16, the attachment direction detection unit 30 determines whether a change event in the attachment direction has been detected (S20). For example, when the subject P intentionally changes the attachment direction during the monitoring target motion, or when the attachment direction of the sensor 200 unintentionally changes during the monitoring target motion, a change event of the attachment direction is detected. Specifically, the attachment direction detection unit 30 may determine that a change event in the attachment direction has been detected when the difference in the attachment direction before and after the difference, that is, the difference in the attachment angle θ₁, is equal to or greater than a predetermined threshold. In this case, the detection of the attachment direction may be performed in the same manner as the detection of the initial attachment direction. Alternatively, the attachment direction detection unit 30 may detect an event of a change in the attachment direction from a change in the sensing-related information with time. For example, when a discontinuous change of a predetermined threshold value or more is detected in the time-series information of the sensing-related information, the attachment direction detection unit 30 may determine that a change event of the attachment direction has been detected. Whether the change is discontinuous may be determined based on whether the difference before and after the sensing-related information is larger than the predicted value by a predetermined threshold or more.

When the attachment direction detection unit 30 determines that an attachment direction change event has been detected (Yes in S20), the process proceeds to S21. Otherwise (No in S20), the process proceeds to S17.

In S21, the control processing unit 32 updates the attachment direction of the sensor 200 associated with the sensing-related information to the attachment direction after the change event. Then, the control processing unit 32 proceeds to S17.

As described above, according to the third embodiment, the motion state monitoring device 3 detects a change during measurement of the attachment direction of the sensor 200, and outputs the changed attachment direction in association with the sensing-related information. Therefore, the motion state monitoring device 3 can manage the subsequent measurement result in association with the changed attachment direction even if the attachment direction is intentionally or unintentionally changed in the middle of the monitoring target motion. Also in the third embodiment, the same effects as those of the first or second embodiment are obtained.

The present disclosure is not limited to the above embodiments, and can be modified as appropriate without departing from the spirit. For example, other embodiments include the following embodiments.

### Other First Embodiments

In the first embodiment, the control processing unit 32 of the motion state monitoring device 3 outputs the sensing-related information in association with the attachment direction of the sensor 200 relative to the reference direction D. However, the control processing unit 32 may convert the relative attachment direction detected by the user into an absolute direction and output the sensing-related information in association with the absolute direction instead of or in addition to the attachment direction.

For example, the control processing unit 32 can calculate the attachment angle θ₁′ between the initial measurement axis A and the Z_{S}-axis by adding the angle θ₀ between the initial reference direction D and the Z_{S}-axis shown in FIG. 3 to the detected initial attachment direction θ₁ of the sensor 200. Then, the control processing unit 32 outputs the attachment angle θ₁′ at the initial stage in association with the sensing-related information as information indicating the absolute orientation of the sensor 200 at the initial stage. The absolute direction of the sensor 200 during measurement can be calculated based on the absolute direction of the sensor 200 at the initial stage, the rotation angle of the sensor 200 which is the measurement result of the sensor 200, and the amount of change in the attachment angle during measurement. In this way, the user can analyze the measurement result in consideration of a more detailed measurement condition, and the analysis accuracy is improved.

### Other Second Embodiments

In the second embodiment, the control processing unit 32 of the motion state monitoring device 3 executes arithmetic processing according to the attachment direction with respect to the sensing information or the sensing-related information. Alternatively or additionally, however, the control processing unit 32 may execute arithmetic processing corresponding to the absolute direction of the sensor 200 described above with respect to the sensing information or the sensing-related information. In this case, the arithmetic processing table 320 may associate the attachment angle θ₁′ described in the other second embodiments with the arithmetic calculation parameters of the arithmetic processing determined in accordance with the attachment angle θ₁′. Thus, the control processing unit 32 can easily compare and use the measurement results regardless of the orientation of the sensor 200.

Although the present disclosure is described above as a hardware configuration in the above embodiments, the present disclosure is not limited to this. In the present disclosure, each process of the motion state monitoring method may be implemented by causing a processor to execute a computer program such as a motion state monitoring program.

In the above embodiments, the computer is a computer system including a personal computer, a word processor, etc. However, the present disclosure is not limited to this, and the computer may be a LAN server, a computer (personal computer) communication host, a computer system connected to the Internet, etc. Functions may be distributed among devices on a network and the computer may be the entire network.

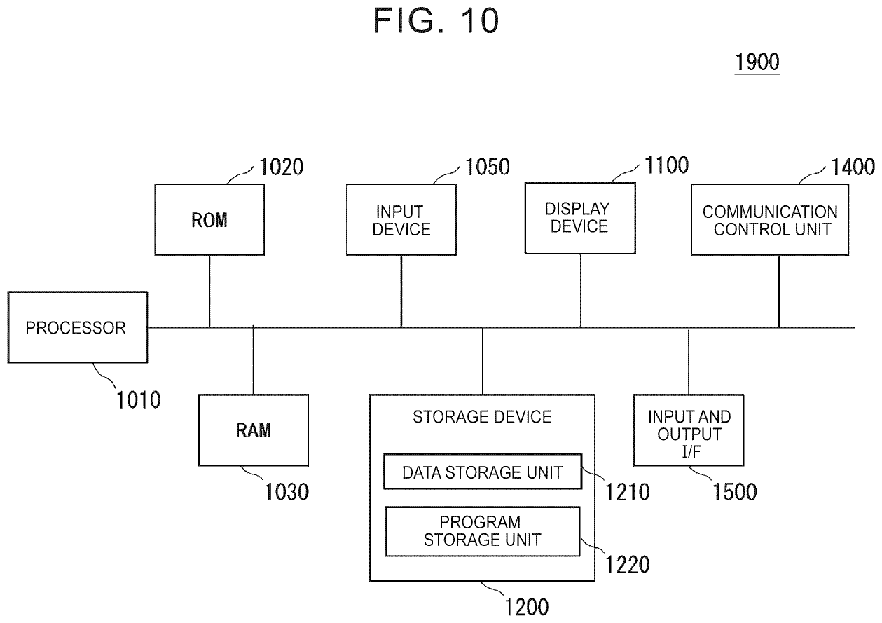

FIG. 10 is a schematic configuration diagram of a computer 1900 according to the above-described embodiment. The computer 1900 includes a processor 1010, a ROM 1020, a RAM 1030, an input device 1050, a display device 1100, a storage device 1200, a communication control device 1400, and an input and output I/F 1500. These are connected via a bus line such as a data bus.

The processor 1010 implements various kinds of control and calculation according to programs stored in various storage units such as the ROM 1020 and the storage device 1200. The processor 1010 may be CPU (Central Processing Unit), GPU (Graphics Processing Unit), FPGA (Field-Programmable Gate Array), DSP (Digital Signal Processor), and ASIC (Application Specific Integrated Circuit).

The ROM 1020 is a read-only memory in which various programs and data for the processor 1010 to perform various controls and calculations are stored in advance.

The RAM 1030 is a random access memory used as a working memory for the processor 1010. In this RAM1030, various areas for performing various processes according to the above embodiments can be secured.

The input device 1050 is an input device that receives input from a user, such as a keyboard, a mouse, and a touch panel.

The display device 1100 is a display that displays various screens under the control of the processor 1010. As the display device 1100, a liquid crystal panel, an organic EL (Electroluminescence), an inorganic EL, etc. may be used. The display device 1100 may be a touch panel that also serves as the input device 1050.

The storage device 1200 is a storage medium having a data storage unit 1210 and a program storage unit 1220. The program storage unit 1220 stores a program for realizing various processes in the above-described embodiment. The data storage unit 1210 stores various data of the various databases according to the above-described embodiments. The storage medium of the storage device 1200 may be a non-transitory computer-readable medium. The non-transitory computer-readable medium includes various types of tangible recording media (tangible storage media). Examples of the non-transitory computer-readable medium include magnetic recording media (e.g., flexible disks, magnetic tapes, hard disk drives), magneto-optical recording media (e.g., magneto-optical disks), CD-ROM (Read Only Memory), CD-R, CD-R/W, DVD (Digital Versatile Disc), BD (Blu-ray (registered trademark) Disc), semiconductor memories (e.g., mask ROM, PROM (Programmable ROM), EPROM (Erasable PROM), flash ROM, and RAM (Random Access Memory)). The storage medium used for storage device 1200 may be various types of transitory computer-readable media. Examples of the transitory computer-readable medium include electrical signals, optical signals, and electromagnetic waves. The transitory computer-readable medium can supply various programs to a computer via a wired communication path such as an electric wire and an optical fiber, or a wireless communication path.

When executing various processes, the computer 1900 reads the program from the storage device 1200 into the RAM 1030 and executes the program. However, the computer 1900 can also execute the program by directly reading the program from an external storage medium into the RAM 1030. In addition, depending on the computer, various programs etc. may be stored in the ROM 1020 in advance, and the processor 1010 may execute the program etc. The computer 1900 may download and execute various programs and data from other storage media via the communication control device 1400.

The communication control device 1400 is a control device for establishing a network connection between the computer 1900 and another external computer. The communication control device 1400 enables access to the computer 1900 from these external computers.

The input and output I/F 1500 is an interface for connecting various input and output devices via parallel ports, serial ports, keyboard ports, mouse ports, etc.

The order of execution of each process in the device and method shown in the claims, the specification, and the drawings may be implemented in any order unless otherwise specified as “before.” “prior to,” or the like, and the output of the previous process is used for subsequent processing. Even when the operation flow in the claims, the specification, and the drawings are described using “first.” “next.” and the like for convenience, the operation flow is not necessarily performed in this order.

What is claimed is:

1. A motion detection system comprising:
a sensor configured to be attached to a user and to detect a motion of the user;
a display unit configured to display an avatar that moves in conjunction with an output of the sensor; and
a determination unit configured to determine that the sensor is not properly prepared when the displayed avatar is deviated from a reference state although the user is in the reference state.

2. The motion detection system according to claim 1, wherein the determination unit is configured to make a determination after the sensor is calibrated.

3. The motion detection system according to claim 1, wherein the sensor being not properly prepared is that an attachment position of the sensor is displaced or that the sensor needs to be recalibrated.

4. The motion detection system according to claim 1, wherein when the determination unit determines that the sensor is not properly prepared, a notification is made by voice, text display on the display unit, or highlighted display on the display unit.

5. A motion detection method comprising:
attaching a sensor to a user and detecting a motion of the user;
displaying, on a display unit, an avatar that moves in conjunction with an output of the sensor; and
determining, by a determination unit, that the sensor is not properly prepared when the displayed avatar is deviated from a reference state although the user is in the reference state.
